(19) United States
(12) Patent Application Publication
(10) Pub. No.: US 2018/0021591 A1
(43) Pub. Date: Jan. 25, 2018

(54) METHOD AND SYSTEM FOR REDUCING FAT CELLS IN A HUMAN BODY

(71) Applicant: Savvy Chic Management INC, DBA The Sculpting Shoppe, Frisco, TX (US)

(72) Inventor: Jessica CHANDLER, Frisco, TX (US)

(73) Assignee: Savvy Chic Management INC, DBA The Sculpting Shoppe, Frisco, TX (US)

(21) Appl. No.: 15/463,334

(22) Filed: Mar. 20, 2017

Related U.S. Application Data
(60) Provisional application No. 62/364,889, filed on Jul. 21, 2016.

(51) Int. Cl. A61N 5/06

(52) U.S. Cl. CPC A61N 5/0616

(57) ABSTRACT

According to an exemplary embodiment, a method and system of non-surgical, non-invasive body contouring and weight reduction treatments may be shown and described. These treatments may provide tissue remodeling, such as skin tightening and skin contour smoothening, as well as provide other cosmetic applications. The method and system may include determining an area of tissue suitable for a noninvasive cosmetic treatment, targeting the area with radiant energy, stimulating the area with a series of micro-pulses, and massaging the area with a vibrational plate. The system and method may result in the reduction of undesired fat deposits and correction of other skin conditions, such as loose skin and cellulite. Furthermore, all aspects of the present method and system may be performed in a non-invasive manner.

### CROSS-REFERENCE TO RELATED APPLICATIONS

This application claims priority from U.S. Provisional Patent Application No. 62/364,889, filed on Jul. 21, 2016, entitled “Method and System to Reduce Fat Cells in a Human Body,” the content of which is hereby incorporated by reference in its entirety.

### BACKGROUND

There is an increasing desire to alter the physical appearance of the human figure and in particular, to eliminate unwanted fatty tissue. Adipose tissue, or fat, is a loose connective tissue that specializes in the storage of triglycerides (fats) within a structural network of fibers. There are two types of adipose tissue found in mammals, namely, white adipose tissue (WAT) and brown adipose tissue (BAT). While brown adipose tissue (BAT) actually contributes to energy expenditure by generating body heat, white adipose tissue (WAT) forms the vast majority of human adipose tissue and specializes in lipid storage.

Overweight bodies and obesity result from excessive fat accumulation primarily in the adipocytes of the WAT. To facilitate an increase in body mass, WAT undergoes pathological expansion. This change occurs as a function of both adipocyte size (hypertrophy) and frequency (hyperplasia). In the face of overnutrition, adipose deposits expand first by hypertrophy until a critical threshold is reached (approximately 0.7-0.9 μg/cell). When demand for lipid storage exceeds the capacity of existing adipocytes, precursor cells (preadipocytes) multiply and differentiate into functioning adipocytes capable of storing and releasing energy. While adipose tissue typically accounts for 20% of body weight in men and 28% of body weight in women, this percentage can increase to upwards of 80% with the proliferation of adipocyte cell count.

Adipose tissue has been known to play an important role in the biological drive to gain weight. While sustained weight loss reduces average adipocyte size and shifts the distribution of cell frequency, once created, the number of adipocytes in the body remains overwhelming consistent throughout adulthood even after dieting and exercise. Excess body fat may be diffuse or may alternatively be concentrated in a particular region of the body. In particular, hard-to-target fat deposits tend to accumulate in the abdomen, buttocks, chest, thighs, arms, and/or chin. These local accumulations can detract from physical appearance, contribute to undesirable body contours, reduce athletic performance, and increase the risk of many health problems, including heart disease, stroke, diabetes, depression and certain cancers.

A variety of surgical and non-surgical methods have been utilized to alter the human body in a desired fashion. General weight loss can be accomplished by diet and exercise, for example. However, there are certain conditions which cannot be addressed or remedied by diet and exercise alone, such as undesired and hard-to-target fat deposits, loose skin, cellulite and the like.

More invasive techniques, such as plastic surgery and liposuction, are often thought of as remedies to some of these problems. Liposuction, for instance, involves the surgical extraction of subcutaneous fat from selected portions of a patient's body. A cannula is inserted through an incision in patient's skin to a space occupied by unwanted excess fat. The cannula is then manipulated in a back and forth motion to mechanically break up and loosen the fatty tissue. The dislodged fat is subsequently aspirated through a central lumen in the cannula by vacuum pressure from a syringe or pump. While effective in removing fatty tissue, the procedure may involve multiple incisions and require many passes of the liposuction cannula in each incision to achieve the desired cosmetic effect for the patient. Moreover, as with any surgical treatment, there are inherent risks beyond the temporary and minor adverse effects. Adverse reactions to anesthesia, deep vein thrombosis, organ perforation, excessive bleeding, risk of infection, surgical and post-surgical pain, and scarring are some common issues and concerns. Additionally, these methods do not guarantee a desired outcome, usually require lengthy recover periods, and are sometimes cost-prohibitive.

There is, therefore, a need for a new method and system to eliminate unwanted fatty deposits in hard-to-target areas of the body without the pain, recovery period, and trauma associated with conventional techniques.

### SUMMARY

According to an exemplary embodiment, a method and system of non-surgical, non-invasive body contouring and weight reduction treatment may be shown and described. The treatment may provide tissue remodeling, such as skin tightening and skin contour smoothening, as well as provide other cosmetic applications. The method and system may include determining an area of tissue suitable for a noninvasive cosmetic treatment, targeting the area with radiant energy, stimulating the area with a series of micro-pulses, and massaging the area with a vibrational plate. The system and method may result in the reduction of undesired fat deposits and correction of other skin conditions, such as loose skin and cellulite. Furthermore, all aspects of the present method and system may be performed in a non-invasive manner.

### BRIEF DESCRIPTION OF THE FIGURES

Advantages of embodiments of the present invention will be apparent from the following detailed description of the exemplary embodiments. The following detailed description should be considered in conjunction with the accompanying figures in which:

FIG. 1 is an exemplary flow chart of a method and system for reducing fat cells in a human body.

### DETAILED DESCRIPTION

Aspects of the invention are disclosed in the following description and related drawings directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiments are not necessarily to be construed as preferred or advantageous over other embodiments. Moreover, the terms “embodiments of the invention”, “embodiments” or “invention” do not require that all embodiments of the invention include the discussed feature, advantage or mode of operation.

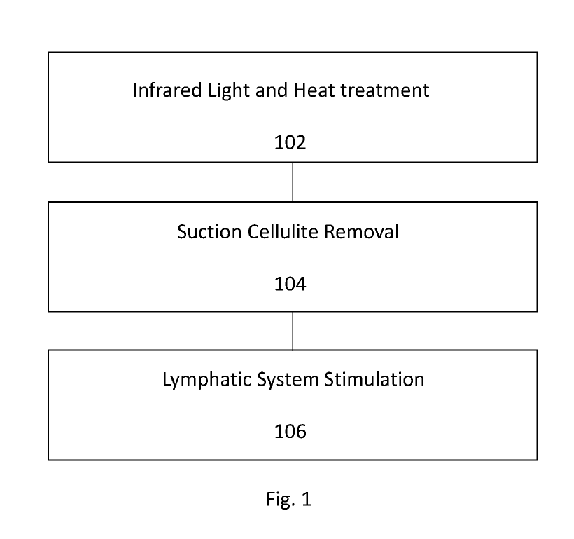

Generally referring to FIG. 1, a method and system for reducing fat cells (adipocytes) in a human body may be shown and described. The present invention may involve a non-surgical, non-invasive body contouring and weight reduction method, whereby a combination of light therapy, micro-pulse stimulation and applied vibration specifically disrupt and ameliorate unwanted fat deposits. The method can generally involve a series of treatment steps, for example exposing a treatment area to radiant energy, massaging a tissue surface of the treatment area, and applying vibrational stimulation to the treatment area. Additionally, the method can utilize a series of components and devices. These devices can include, but are not limited to, a light emitting diode (LED) system that provides low level light therapy, a massage appliance operating by suction and mobilization of skin tissue, and a vibration platform.

Still referring to exemplary FIG. 1, light treatment 102 may be performed on a desired portion of the body. The treatment area may be a location of the body having unwanted fat accumulation and/or skin laxity, such as the back, stomach, neck, arms, buttocks, thighs, ankles and chest. The light treatment may utilize red and/or near infrared light (600 nm-1000 nm) to affect cell membrane permeability. The treatment 102 may be performed by a light-emitting device having a series of conducting pads associated therewith. The conducting pads may be releasably coupled, via an adhesive or physical coupling, to an area of the body to be treated. Each conducting pad may be cylindrically-shaped and have a front surface and a rear surface. The front surface may include a plurality of light terminations and be positioned proximate the skin in order to permit uninterrupted emission of light to the treatment area. The plurality of light terminations may be arranged in a pattern that promotes full irradiation of the treatment area underneath. The number of conducting pads employed during treatment may depend upon the expanse of the treatment area. Treatment to the stomach and back, for example, may require the circumferential application of eight conducting pads arranged in series around the midsection.

In an exemplary embodiment, the light-emitting device may include sixteen conducting pads, each with an area of 16×110×65 mm. Each conducting pad may have twenty-eight light source terminations rigidly mounted to a front surface. The conducting pads may be securely fastened to a treatment area with a length-adjustable strap that holds the front surface of each conducting pad directly against the patient's skin. The light source terminations may emit light at a wavelength between about 630 and 640 nm with a power density of 500 mW per pad (measured at the source).

Each conducting pad may be communicatively coupled, for example via electrical wiring, to a data processing unit for regulating the power and emitting sequence of the light source. The data processing unit may be a programmable device that responds to a specific set of instructions and can execute the set of instructions accordingly. For example, the data processing unit may control the time and energy emitted from each of the conducting pads. The data processing unit may include at least one non-transitory computer-readable medium storing executable instructions, a central processor adapted to execute the instructions, a user interface with interactive display (e.g., LCD screen) for operating the system, and other associated electronic circuitry as would be known and understood by a person having ordinary skill in the art. The data processing unit may be connectable to a power source via electrical wiring.

The user interface of the data processing unit may feature a plurality of operating modules that regulate different functionalities of the data processing unit. For example, the user interface may include a key switch to turn the device on and off, interval selection buttons to specify a duration of treatment, a display panel to present treatment time, media control buttons to start/pause/stop treatment progress, a pad selector to activate a predetermined number of conducting pads, and a safety button to provide emergency-stop capabilities. The desired treatment area may determine the extent of treatment as it relates to the number of activated conducting pads, and duration of light exposure.

Still referring to step 102, a desired and predetermined amount of light and/or heat may be provided to a treatment area through the conducting pads of the light-emitting device. For example, light with a wavelength of about 635 nm may be provided through light terminations mounted on a front surface of the conducting pads. The light terminations may be coupled to a light source. It may be appreciated that the light source may be laser-based, LED-based or any desired type of light source, as would be understood by a person having ordinary skill in the art. Further, the light source may include a single source, multiple sources, or any combination of any desired sources, as may be understood by a person having ordinary skill in the art. Additionally, heating elements disposed in the conducting pads may give off a desired temperature range of heat to the treatment area. It should be noted that the heat may be set in a predetermined range so as to promote therapeutic effectiveness and reduce discomfort, while also avoiding any burning sensation or actual burning of the skin.

The light from the light-emitting device may penetrate below the skin surface and into the subdermal tissues of the treatment area at a depth sufficient to stimulate lipolysis in the adipocytes. The light may be absorbed into the cytochrome-c-oxidase unit of the proton transport chain within the mitochondria, facilitating a temporary imbalance of charge distribution within the cell walls and stimulating the production of cytoplasmic lipase. Cytoplasmic lipase may convert stored triglycerides into fatty acids and glycerol, which can both pass through pores formed in the cell membrane thereby causing a shrinkage in adipocytes and promoting removal or elimination of the adipocytes from the body. Lipolysis of the adipocytes may occur once the light-emitting device delivers a sufficient energy density to the adipose tissue layer of the treatment area.

In another exemplary embodiment, the light treatment 102 may be accomplished through the use of an infrared sauna. The infrared sauna may include a sauna chamber having a floor, a plurality of interior walls having at least one door disposed therein, and a ceiling that forms a personal enclosure. The chamber may further include one or more seating structures configured to support a human body, such as a chair, bench, or other seating platform. The seating structure may be arranged within the chamber to maximize exposure of the treatment area to infrared therapy.

The sauna chamber may be equipped with a number of infrared emitters for directing infrared radiation, experienced as radiant heat, to a human body situated therein. The emitters may be distributed about the interior of the chamber, installed within the interior walls or alternatively mounted thereto. The emitters may be evenly spaced around the chamber, or may be distributed in a predefined pattern. It should be appreciated by those skilled in the art that the positioning of infrared emitters can be variable based on the application and desired therapeutic effect.

The infrared emitters may be configured to emit infrared radiation over specified wavelength and frequency ranges to help promote resonant absorption. In some exemplary embodiments, the infrared emitters may emit infrared radiation at any wavelength within the infrared wavelength spectrum, such as, for example, near infrared, mid infrared, or far infrared. In other exemplary embodiments, the infrared emitters may emit radiation in a specified wavelength range, for example, from about 7-14 microns. The infrared emitters may be constructed of ceramic, carbon, and/or infrared light-emitting diodes (LED); however, it should be appreciated by those skilled in the art that the infrared emitters may be made from any suitable material known within the art. The infrared energy may be able to penetrate up to five centimeters beneath the skin surface. The therapeutic benefits associated with the penetration of infrared energy may include, but are not limited to, the release of toxins stored within subcutaneous fatty deposits, accelerated weight loss, skin rejuvenation, and improved circulation, cardiovascular function and lymphatic flow.

The infrared sauna may be communicatively coupled, for example via electrical wiring, to a data processing unit for regulating the interior environment of the sauna chamber. The data processing unit may control, for example, the power, intensity, duration and emitting sequence of the infrared emitters. The data processing unit may be a programmable device that responds to a specific set of instructions and can execute the set of instructions accordingly. The data processing unit may include at least one non-transitory computer-readable medium storing executable instructions, a central processor adapted to execute the instructions, a user interface with interactive display (e.g., LCD screen) for operating the system, and other associated electronic circuitry as would be known and understood by a person having ordinary skill in the art. Each infrared emitter may be independently controlled so as to allow selective and customizable application of infrared radiation. All controlled features may also have independent controls. In one exemplary embodiment, the data processing unit may regulate the internal temperature of the sauna chamber within a range from about 90 to 200 degrees Fahrenheit. The controlled features may be preprogrammed prior to each treatment session, or may be user-manipulatable throughout the duration of treatment.

Still referring to step 102, the light treatment can be applied for any desired amount of time, for example 20 minutes or any time range from about 5 minutes to about 30 minutes, and may utilize multiple sessions to continue providing desired effects. In an exemplary embodiment, the treatment 102 may be performed twice a week over a four-week period, totaling eight sessions overall. In another exemplary embodiment, the treatment 102 may be performed twice a week over a four-week period, and then once a week over an additional two-week period. It should be noted that the number of treatments 102 can vary depending on the specified treatment area and/or desired treatment results.

Referring now to step 104, after the light treatment 102, cellulite reduction and removal may take place with a cellulite treatment system for massaging the human body. The cellulite treatment system may employ an adjustable massage device that softens the subcutaneous tissue of the treatment area, via mechanical stimulation, in order to release excesses and smooth the skin. The massage device may be able to apply different massage techniques to the treatment area, such as rolling, lifting, kneading, squeezing, vibrating, and the like. An exemplary device that may be utilized in this step may be the commercial product Wellbox®. The device may be applied to the same areas targeted in the previous step 102.

The massage device in step 104 may provide a series of micro-pulses to the skin, thereby activating cellular regeneration and triggering lipolysis of adipocytes. These micro-pulses may stimulate the skin through suction, compression, and decompression, as well as targeting fibroblast cells. As fibroblast cells lead to the generation of collagen and elastin which, in turn, cause cellulite, the stimulation of the fibroblast cells can have the effect of reducing and eliminating the undesired cellulite in the targeted areas. For example, the method described herein may cause a smoothing of cellulite that effectively removes its appearance. Additionally, through rolling and suction action, skin and cells in the targeted areas can be compressed and decompressed, causing the release of the fat cells which were previously treated and affected with the light and/or heat treatment 102 described above.

The massage device utilized in step 104 may include a treatment head with independent, motorized rollers positioned in series along a bottom portion thereof. The rollers, or any other type of spherical compression surface as known or desired, may be rotatably mounted in a chamber and moveable over the treatment area. The rollers can be rotationally driven by a motor via a drive transmission. A vacuum or other type of air transfer duct may be communicatively coupled to the chamber, so that as the rollers are moved against the skin surface, suction from the vacuum causes a fold of skin to be pulled up into the chamber and between the rollers. The rollers may be interchangeable having widths of 15 mm, 30 mm, and 45 mm; use of roller width may depend on the size of the treatment area. A push button or switch may be located on the treatment head to control the rotation of the rollers in either a forward or backward direction.

Operation of the massage device may be directed by a control unit. The control unit may be a programmable device that responds to a specific set of instructions and can execute the set of instructions accordingly. The control unit may include at least one non-transitory computer-readable medium storing executable instructions, a central processor adapted to execute the instructions, a user interface with interactive display (e.g., LCD screen) for operating the system, and other associated electronic circuitry as would be known and understood by a person having ordinary skill in the art. The control unit may be connectable to a power source via electrical wiring.

The user interface of the control unit may include a plurality of operating modules and an LCD screen that displays the settings to adjust based on the treatment to be performed. The operating modules may include a power key to select and adjust a desired intensity level on a scale from one through six, a rhythm key to select a treatment program (e.g., no rhythm, low rhythm, medium rhythm or high rhythm), a time key to define the session duration, and an operation key to start, stop, continue, or cancel a treatment session.

In an exemplary embodiment, the massage device can be positioned with the rollers against the skin surface of the treatment area, for example the abdomen. The progression of treatment should begin with the uppermost portion of the treatment area, in this instance the upper abdomen or epigastric and hypochondriac regions. The massage device may be programmed to operate at an intensity level of five, medium rhythm, and for a duration of six minutes. After the setting adjustment, the massage device may be turned on and manually shifted from left hypochondriac region to right hypochondriac region and vice versa for approximately three minutes. The massage device may then be positioned against the lower abdomen, hypogastric and iliac regions, and moved side to side for the remaining three minutes. The entire six-minute treatment session (e.g., upper and lower abdomen) may be repeated after an adjustment of the massage device settings to an intensity level of four with no rhythm. It may be appreciated that the settings can be adjusted based on the treatment area and desired effect of treatment. The cellulite reduction and removal step 104 can be applied for any desired amount of time, for example 20 minutes or any time range from about 5 minutes to about 30 minutes, and may utilize multiple sessions to continue providing desired effects.

Referring now to step 106 of exemplary FIG. 1, vibrations and massaging may be utilized to stimulate the lymphatic system, further effecting the reduction and removal of undesired fat cells. A vibration plate, or other similar vibration or stimulation device, may then be utilized following the completion of steps 102 and 104. The vibration plate may stimulate the lymphatic system, resulting in the reduction or elimination of undesired fat cells in the targeted area. As the lymphatic system is generally responsible for the transportation of fatty acids and fats throughout the body for metabolism, the stimulation of the lymphatic system further promotes the movement of the affected fat cells in the areas of the body targeted in steps 102 and 104. In general, areas of the body with high fat cell counts can have impaired lymphatic circulation and drainage, leading to conditions related to cellulite, fluid retention, fat pockets, lumpy skin, and the like. Thus, once the fat cells are treated as above, the stimulation of the lymphatic system can encourage removal of the fat cells from the targeted areas. The vibration plate may be a plate that a user stands on and which vibrates the entire body, for example for about ten minutes. Alternatively, the vibration plate may be a device that may be applied to targeted areas of the body for approximately ten minutes, although it is envisioned any other duration of time may be utilized, as desired for either a vibration plate that is stood upon or which is applied to targeted areas of the body.

As described herein, the method and system may be utilized through a series of treatment steps taken one after the other. It may also be appreciated that multiple treatment sessions involving all three steps may be utilized or desired in order to achieve a desired amount of fat reduction or body sculpting. Further, it is also envisioned that the various steps described herein could be combined into a singular device capable of providing all treatments.

The foregoing description and accompanying figures illustrate the principles, preferred embodiments, and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. A method of treatment for reducing fat cells in a human body, comprising:
(a) providing light therapy to a targeted area of the body;
(b) stimulating the targeted area of the body with a series of micro-pulses; and
(c) massaging the body with a vibration plate thereby promoting lymphatic drainage.

2. The method of claim 1, further comprising performing the light therapy with a plurality of conducting pads, each conducting pad having a plurality of light terminations mounted to a front surface thereof.

3. The method of claim 2, further comprising emitting light at a predetermined wavelength from a light source coupled to the plurality of light terminations of each conducting pad.

4. The method of claim 3, further comprising providing a data processing unit communicatively coupled to the light source and plurality of conducting pads.

5. The method of claim 4, wherein the data processing unit comprises at least one non-transitory computer-readable medium storing executable instructions, a central processor adapted to execute the instructions, and a user interface.

6. The method of claim 5, wherein the user interface comprises a plurality of operating modules that regulate different functionalities of the data processing unit, including a power switch, internal selection buttons to specify a duration of treatment, media control buttons to start, pause, and stop treatment progress, a pad selector to active a predetermined number of conducting pads from the plurality of conducting pads, and a safety button to provide an emergency-stop.

7. The method of claim 2, further comprising coupling the conducting pads to the targeted area of the body via one of an adhesive and physical coupling.

8. The method of claim 3, wherein the light source produces light at a wavelength of 635 nm.

9. The method of claim 2, further comprising applying the conducting pads to the targeted area of the body for a duration of 5-20 minutes.

10. The method of claim 2, wherein the plurality of conducting pads is 16 conducting pads and the plurality of light terminations is 28 light terminations.

11. The method of claim 1, further comprising generating the micro-pulses with a massage device having a treatment head with rollers positioned in series along a bottom portion thereof.

12. The method of claim 11, wherein the rollers are rotatably mounted in a chamber of the massage device and moveable over the targeted area of the body.

13. The method of claim 12, further comprising providing a vacuum in communication with the chamber, so that as the rollers are moved against a skin surface, suction from the vacuum causes a fold of skin to be pulled up into the chamber and between the rollers.

14. The method of claim 11, wherein the rollers are interchangeable having widths of 15 mm, 30 mm, and 45 mm.

15. The method of claim 11, further comprising disposing a push-button on the treatment head of the massage device to control a rotation of the rollers in a forward and backward direction.

16. The method of claim 1, wherein the micro-pulses stimulate the skin through physical manipulation including at least one of rolling, suction, compression, and decompression.

17. The method of claim 11, further comprising operating the massage device with a control unit, wherein the control unit comprises at least one non-transitory computer-readable medium storing executable instructions, a central processor adapted to execute the instructions, and a user interface.

18. The method of claim 17, wherein the user interface comprises a plurality of setting modules that regulate different functionalities of the massage device, including a power key to select a desired intensity level, a rhythm key to select a treatment program, a time key to define a treatment duration, and an operation key to start, stop, continue, and cancel a treatment session.

19. The method of claim 1, wherein a user stands on the vibration plate.

20. The method of claim 1, further comprising applying the vibration plate to the targeted area of the body for a predetermined amount of time.
